(19) United States
(12) Patent Application Publication
(10) Pub. No.: US 2010/0196936 A1
(43) Pub. Date: Aug. 5, 2010

(54) METHOD AND KIT FOR DETECTING CANCER

(75) Inventors: Semon Wu, Hualien (TW); Ming-Cheng Lee, Hualien (TW)

Correspondence Address: EDWARDS ANGELL PALMER & DODGE LLP, P.O. BOX 55874, BOSTON, MA, 02205, US

(73) Assignee: TZU CHI BUDDHIST GENERAL HOSPITAL, Hualien (TW)

(21) Appl. No.: 12/539,898

(22) Filed: Aug. 12, 2009

(30) Foreign Application Priority Data
Feb. 4, 2009 (TW) TW 098103455

(51) Int. Cl. G01N 33/00

(52) U.S. Cl. 435/7.92; 435/29

(57) ABSTRACT

A method for detecting cancer is disclosed. The method includes the steps of: (1) obtaining a test sample of body fluid from a subject; (2) measuring a calsyntenin-1 (CLSTN-1) level in the test sample of body fluid in vitro; and determining if the CLSTN-1 level in the test sample of body fluid is significantly higher than a normal CLSTN-1 level in a sample of body fluid from a healthy population of the same species as the subject, and if yes, indicating that the subject under test has cancer. Methods for monitoring therapeutic efficacy of a therapy or a medicament for treating a cancer patient and for identifying efficacy of a medicament in treatment of cancer are disclosed, and a kit for use in the aforesaid methods is also provided.

### FIELD OF INVENTION

The present invention relates to a method for detecting cancer, a method for monitoring the therapeutic efficacy of a therapy or a medicament for treating a cancer patient, a method for identifying efficacy of a medicament in treatment of cancer, and a kit for use in the aforesaid methods, wherein calsyntenin-1 (CLSTN-1) is used as a biomarker for detection.

### DESCRIPTION OF RELATED ART

Cancer is ranked first among the top 10 causes of death in Taiwan, and is also a worldwide medical issue. Among various kinds of cancers, the lung cancer was the primary cause of death. Further, morbidity and fatality rates of lung cancer in Taiwan rapidly increased. Lung cancer can be classified by cell morphology into small cell lung cancer and non-small cell lung cancer, wherein non-small cell lung cancer is the most common type of lung cancer, and accounts for more than 85% of lung cancer. Thus, early diagnosis and effective treatment of cancer, especially lung cancer, have become important subjects of investigation.

Early detection of cancer is a critical factor for successfully treating cancer and increasing survival rates of patients. There are various types of methods for detecting cancer, such as biomarker detection. In biomarker detection, a sample of a subject's body fluid, such as serum, is tested for the presence of specific protein molecules (biomarker), and/or different concentrations of the biomarker to screen for cancer, or monitor the cancer process, treatment reaction and cancer recurrence. Because biomarker detection has high sensitivity and is applicable to a small sample size, it plays an important role in detecting and monitoring cancer.

Examples of a biomarker commonly used for detecting cancer include, but not limited to, carcinoembryonic antigen (CEA) for detecting breast cancer, large intestine cancer, stomach cancer, prostate cancer, ovarian cancer and lung cancer; and protein molecules of tumor cells such as neuron-specific enolase, carbohydrate antigen 125 (CA-125), and cytokeratin 19 segment (CYFRA 21-1) for use in screening for and monitoring of cancer process.

However, the disadvantage of using a biomarker is that the biomarker used must have specificity, particularly, the expression level of the biomarker in cancer cells must be significantly different from that of the biomarker in normal cells; preferably, the biomarker is highly expressed in a cancer patient but poorly expressed or not expressed in a healthy population. Currently, commonly used biomarkers for cancer screening, such as CEA, have poor sensitivity and specificity.

On the other hand, it is known that calsyntenin-1 (CLSTN-1), which is a type 1 neuronal transmembrane protein on the postsynaptic membrane of the nervous system, can bind with calcium (Vogt et al., 2001). Further, US Patent Application Publication No. 20040019919 discloses that CLSTN-1, 2 and 3 can be used in treating nervous system diseases, particularly central nervous system diseases. Although US Patent Application Publication No. 20040019919 vaguely discloses that CLSTN can be expressed in tumor cells, there is no further discussion on this part. Moreover, there are no other literatures or publications alleging that CLSTN-1 molecules have any correlation with liver cancer to date. Accordingly, there still exists a need to develop biomarkers with high specificity and sensitivity for detecting cancer.

### SUMMARY OF THE INVENTION

The inventors of the present invention analyzed expression levels of secretory proteins of cancer tissues (such as pulmonary adenocarcinoma) of cancer patients by immunohistochemical staining method or Western blot analysis. The results show that the expression levels of CLSTN-1 in the cancer tissues are significantly different from that of CLSTN-1 in normal tissues of a healthy population. Therefore, the inventors of the present invention carried out further investigation, and proved that CLSTN-1 can be used as a biomarker for screening for cancer, such as lung cancer.

An aspect of the present invention is to provide a method for detecting cancer, including the steps of:

(1) obtaining a sample of body fluid from a subject under test;

(2) measuring a CLSTN-1 level in the sample of body fluid in vitro; and

(3) determining if the CLSTN-1 level in the sample of body fluid is significantly higher than a normal CLSTN-1 level in a sample of body fluid from a healthy population of the same species as the subject under test, and if yes, indicating that the subject under test has cancer.

Another aspect of the present invention is to provide a method for monitoring the therapeutic efficacy of a therapy or a medicament for treating a cancer patient, including the steps of:

(1) obtaining samples of body fluid from the cancer patient at one or more time points before and after applying the therapy or the medicament;

(2) measuring CLSTN-1 levels in the samples of body fluid in vitro;

(3) comparing the CLSTN-1 levels in the samples of body fluid to one another; and

(4) determining if the CLSTN-1 levels in the samples of body fluid after applying the therapy or the medicament are significantly lower than the CLSTN-1 levels in the samples of body fluid before applying the therapy or medicament, and if yes, indicating that the therapy or the medicament is effective in the treatment of the cancer patient.

Another aspect of the present invention is to provide a method for identifying a medicament efficacy in treatment of cancer, including the steps of:

(1) selecting a group of mammals having cancer;

(2) obtaining samples of body fluid from the mammals at one or more time points before and after applying the medicament;

(3) measuring CLSTN-1 levels in the samples of body fluid; and

(4) determining if the CLSTN-1 levels in the samples of body fluid after applying the medicament are significantly lower than the CLSTN-1 levels in the samples of body fluid before applying the medicament, and if yes, indicating that the medicament is effective in the treatment of cancer.

Another aspect of the present invention is to provide a kit for use in any of the aforesaid methods, wherein the kit includes a molecule capable of recognizing CLSTN-1 or unique segment(s) thereof, and a container or a solid support for carrying the molecule. In a preferred embodiment of the present invention, the kit may optionally include a reagent for detecting the molecule capable of recognizing CLSTN-1 or unique segment(s) thereof and/or an instruction booklet for using the kit.

A further aspect of the present invention is to provide the application of CLSTN-1 as a biomarker in a method for detecting cancer in a subject, a method for monitoring the therapeutic efficacy of a therapy or a medicament for treatment of cancer, and a method for identifying efficacy of a medicament in treatment of cancer.

### BRIEF DESCRIPTION OF THE DRAWINGS

FIG. 1 is an image showing expression levels of CLSTN-1 in a lung tissue array of non-small cell lung cancer patients detected by an immunohistochemical staining method, wherein (A) denotes samples of normal lung tissues, (B) denotes samples of stage I lung cancer tissues, (C) denotes samples of stage II lung cancer tissues, and (D) denotes sample of stage III lung cancer tissues;

FIG. 2A is a histogram showing expression levels of CLSTN-1 by Western blot analysis, wherein the reference numeral ‘1’ denotes serum sample from non-small cell lung cancer patient, and the reference numeral ‘2’ denotes serum sample from healthy volunteer;

FIG. 2B is a histogram showing the expression levels of CEA by Western blot analysis, wherein the reference numeral ‘1’ denotes serum sample from non-small cell lung cancer patient, and the reference numeral ‘2’ denotes serum sample from healthy volunteer;

FIG. 3 is an image showing expression levels of CLSTN-1 in serum samples of non-small cell lung cancer patients obtained in different time points detected by Western blot analysis; and

FIG. 4 is an image showing expression levels of CLSTN-1 in a tissue array of melanoma detected by an immunohistochemical staining method, wherein (A) denotes samples of normal skin tissues, and (B) denotes samples of stage II melanoma.

### DETAILED DESCRIPTION OF THE INVENTION

The present invention provides a method for detecting cancer in a subject, including the steps of:

(1) obtaining a sample of body fluid from the subject;

(2) measuring a CLSTN-1 level in the sample of body fluid in vitro; and

(3) determining if a measured CLSTN-1 level in the sample of body fluid is significantly higher than a normal CLSTN-1 level in a sample of body fluid from a healthy population of the same species as the subject, and if yes, indicating that the subject has cancer.

Cancers that can be detected by the method of the present invention are those having abnormally high expression levels of CLSTN-1 in tissues, and the type of cancer can be, but not limited to, lung cancer, skin cancer, or the like.

In the aforesaid method of the present invention, the subject is usually a mammal, including, but not limited to, human, rat, mouse, rabbit, monkey, cattle, pig, cat, dog and the like, and preferably human.

In the aforesaid method of the present invention, the sample of body fluid is preferably blood, and more preferably serum.

In the aforesaid method of the present invention, the sample of body fluid can be tested by any conventional protein-detecting methods. Generally speaking, the sample of body fluid can be tested by enzyme immunoassay or Western blot analysis. In these methods, the molecules used for recognizing CLSTN-1 or unique segment(s) thereof must be able to bind to them specifically. Therefore, CLSTN-1 antibodies, and preferably CLSTN-1 monoclonal antibodies are used.

In the aforesaid method of the present invention, the term “healthy population” refers to a population that is the same species as the subject, and does not have cancer. The term “normal CLSTN-1 level” refers to the average CLSTN-1 level of all the samples of body fluid obtained from the healthy population. Generally speaking, CLSTN-1 is rarely expressed or very poorly expressed in non-cancer cells.

In the aforesaid method of the present invention, the expression “significantly higher” is defined as the CLSTN-1 level of a sample of body fluid of a subject under test being 5 or more times higher than a normal CLSTN-1 level. If the CLSTN-1 level in the sample of body fluid of the subject is significantly higher than the normal CLSTN-1 level, the subject under test has cancer.

In the aforesaid method of the present invention, CLSTN-1 can also be used in combination with other biomarkers to improve the accuracy of detection. Further, the method of the present invention for detecting cancer in a subject can be used in combination with other methods.

It can be understood from the following experimental examples that the severity degree of cancer is positively correlated to the CLSTN-1 level. Accordingly, the present invention also provides a method for monitoring the therapeutic efficacy of a therapy or a medicament for treating a cancer patient, including the steps of:

(1) obtaining samples of body fluid from the cancer patient at one or more time points before and after the therapy or the medicament is applied;

(2) measuring CLSTN-1 levels in the samples of body fluid in vitro;

(3) comparing measured CLSTN-1 levels to one another; and

(4) determining if the measured CLSTN-1 levels in the samples of body fluid after applying the therapy or the medicament are significantly lower than those in the samples of body fluid before applying the therapy or medicament, and if yes, indicating that the therapy or the medicament is effective in the treatment of the cancer patient.

The invention further provides a method for identifying efficacy of a medicament in treatment of cancer, including the steps of:

(1) selecting a group of mammals having cancer;

(2) obtaining samples of body fluid from the mammals at one or more time points before and after the medicament is applied;

(3) measuring CLSTN-1 levels in the samples of body fluid; and

(4) determining if the measured CLSTN-1 levels in the samples of body fluid after applying the medicament are significantly lower than those in the samples of body fluid before applying the medicament, and if yes, indicating that the medicament is effective in the treatment of cancer.

The present invention further provides a kit for use in any of the aforesaid methods. The kit includes a molecule capable of recognizing CLSTN-1 or unique segment(s) thereof, and a container or a solid support for carrying the molecule. In a preferred embodiment of the present invention, the kit may optionally include a reagent for detecting the molecule capable of recognizing CLSTN-1 or unique segment(s) thereof, and/or an instruction booklet for using the kit.

In the kit of the present invention, the term “unique segment(s) thereof” refers to the segments present only in CLSTN-1 but not in other proteins or peptides. In the kit of the present invention, the molecule recognizing CLSTN-1 or unique segment(s) thereof must be able to bind specifically thereto. Generally speaking, CLSTN-1 antibodies, and preferably CLSTN-1 monoclonal antibodies, are used. The CLSTN-1 antibodies can be prepared by conventional antibody-preparing methods.

The molecules used for recognizing CLSTN-1 or unique segment(s) thereof can be in solid form or in liquid form by dissolving or suspending the molecules in solution. The molecules can be carried by a suitable container such as a vial or a solid support, for example, by coating the molecule on a test panel.

In the kit of the present invention, a reagent used for detecting the molecule (e.g. a CLSTN-1 antibody) for recognizing CLSTN-1 or unique segment(s) thereof includes any suitable reagents conventionally used in protein detection methods such as an immunohistochemical staining method, enzyme immunoassay or Western blot analysis, wherein the reagent is such as a fluorescent substance, an enzyme, a substrate, and the like. In a preferred embodiment of the present invention, the molecule that can recognize CLSTN-1 or unique segment(s) thereof is a CLSTN-1 monoclonal antibody or a CLSTN-1 polyclonal antibody, and the reagent for detecting the molecule for identifying CLSTN-1 or unique segment(s) thereof is a CLSTN-1 primary antibody that is linked to horseradish peroxidase or other markers.

The kit of the present invention further includes an instruction booklet, wherein the instruction booklet provides information on test conditions, operating steps and cautions. Persons having ordinary skills in the art should be able to understand the information, and accordingly apply the information to practical implementation.

The present invention also provides an application of CLSTN-1 as a biomarker in a method for detecting cancer, a method for monitoring the therapeutic efficacy of a therapy or a medicament for treatment of a cancer patient; and an application of CLSTN-1 in preparing a biomarker or composition used in the aforesaid methods.

### REFERENCE EXAMPLE 1

### Preparation of CLSTN-1 Antibodies

Peptides consisting of 19 amino acids, which are amino acids No. 561 to No. 579 of human CLSTN-1 sequence, are used as immunogens to immunize rabbits for obtaining CLSTN-1 antiserum.

On day 0, rabbit blood was sampled as a control group before immunization. Then, antigens (250 mg) and complete adjuvant were injected subcutaneously into the rabbit. In weeks 2, 4, 6, 8, and 10, antigen boosts were performed by injecting 150 mg of antigens and incomplete adjuvant each time to the rabbits. In weeks 7, 9 and 11, blood was sampled, and serum was removed to determine antibody titers. After obtaining a desired antibody titer, rabbit blood was sampled again, and the serum was removed to obtain CLSTN-1 antibodies.

### EXPERIMENTAL EXAMPLES

The Experimental Examples of the present invention were performed under the approval of the Human Trial Committee of the Tzu Chi Buddhist General Hospital, Taiwan.

### EXPERIMENTAL EXAMPLE 1

The expression levels of CLSTN-1 in the lung tissue array of non-small cell lung cancer patients were examined by an immunohistochemical staining method.

Commercially available lung cancer tissue array, LUC96101 (purchased from Pantomic, USA), includes 4 samples of stage I non-small cell lung cancer patients, 5 samples of stage II non-small cell lung cancer patients, and 10 samples of ≧stage III non-small cell lung cancer patients. In addition, 4 samples of normal lung tissue array without cancer were used as control groups.

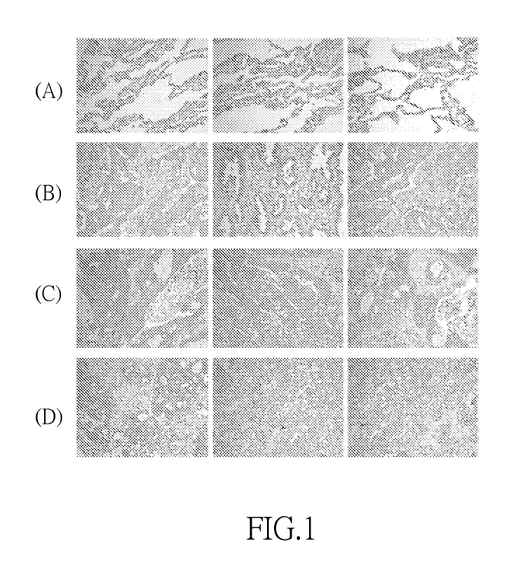

The lung tissue arrays were heated using a microwave, placed in an ethanol solution containing 2% H₂O₂, and fixed by 2% of BSA, 0.1% of Triton X-100 and 1% of sheep serum. The lung tissue arrays and CLSTN-1 monoclonal antibodies were incubated at 37° C. for 2 hours, and horseradish hydroperoxidase (HRP) (purchased from Biogenes, Germany) was added to bind to the CLSTN-1 monoclonal antibodies that formed complexes with the lung tissue arrays at room temperature for 30 minutes, and then 3,3-diaminobenzidine was added for imaging. Results are shown in FIG. 1.

As shown in FIG. 1, CLSTN-1 was expressed strongly and profusely in the cytoplasm of the lung cancer cells, but it was rarely expressed in the cytoplasm of the normal lung cells. Further, the results of the immunohistochemical staining of stages I, II, and III lung cancers indicate that the expression levels of CLSTN-1 are positively correlated with the severity degree of lung cancer.

### EXPERIMENTAL EXAMPLE 2

Expression levels of CLSTN-1 in the serum samples obtained from non-small cell lung cancer patients were detected by Western blot analysis.

In a routine health check, 20 non-small cell lung cancer patients including 10 stages III and 10 stages IV lung cancer patients were selected as subjects under test, and 10 healthy volunteers of the same age/sex were selected to represent the healthy population (the control groups). Blood samples were obtained from the subjects under test and healthy volunteers, and they were centrifuged to obtain serum samples respectively.

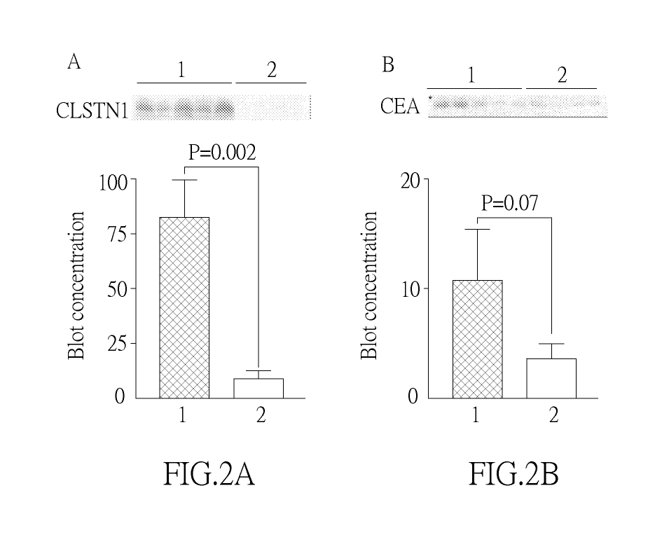

The serum samples obtained were examined by Western blot analysis. Proteins in the serum samples were separated by electrophoresis with a 10% sodium lauryl sulfate-polyvinyl amine gel (SDS-PAGE). The electrophoresed gel was blotted onto a polyvinylidene fluoride (PVDF) film, and treated with CLSTN-1 antibodies (prepared in Reference Example 1) and secondary antibodies (rabbit anti-IgM) linked to HRP for 1 hour. Then, ECL chemiluminescence immunoassay (purchased from Amersham) was performed for imaging. Results were quantified, and shown in FIG. 2A.

Moreover, CEA antibodies (purchased from Thermo Scientific, USA) were added, and CEA in the serum samples were detected by Western blot analysis and quantified. Results are shown in FIG. 2B.

As shown in FIG. 2A, CLSTN-1 was strongly expressed in the serum samples of the lung cancer patients, but it was rarely or poorly expressed in the serum samples of the healthy volunteers. Moreover, the expressions levels of CLSTN-1 in the serum samples of the lung cancer patients were more than 5 times greater than those in the serum samples of the healthy volunteers. Further, the difference between the two groups of serum samples in terms of expression levels of CLSTN-1 was statistically significant (p=0.002).

As shown in FIG. 2B, the difference between the two groups of serum samples in terms of expression levels of conventional CEA biomarkers was statistically significant (p=0.07), but the difference was smaller than that in the previous case of CLSTN-1. In other words, CLSTN-1 is more sensitive than the conventional CEA as a biomarker.

### EXPERIMENTAL EXAMPLE 3

Expression levels of CLSTN-1 in the serum samples obtained from patients with different stages of non-small cell lung cancers were detected by Western blot analysis.

In a routine health check, 15 non-small cell lung cancer patients including 5 stages I, II and III non-small cell lung cancer patients each were selected as subjects under test. Blood samples were obtained from the subjects under test, and they were centrifuged to obtain serum samples respectively.

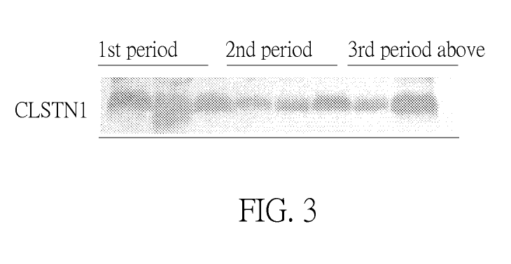

The serum samples obtained were examined by Western blot analysis. Proteins in the serum samples were separated by electrophoresis with 10% SDS-PAGE. The electrophoresed gel was blotted onto a polyvinylidene fluoride (PVDF) film, and treated with CLSTN-1 antibodies (prepared in Reference Example 1) and secondary antibodies (rabbit anti-IgM) linked to HRP for 1 hour. Then, ECL chemiluminescence immunoassay was performed for imaging. Results were quantified, and shown in FIG. 3.

As shown in FIG. 3, CLSTN-1 was detected in all of the samples. Although the expression levels of CLSTN-1 in blood were not positively correlated with the severity degree of lung cancer, the expression levels of CLSTN-1 in patients with stage I non-small cell lung cancer were easily detected. This indicated that the sensitivity of CLSTN-1 is higher than that of the conventional CEA as a biomarker. Hence, CLSTN-1 can be used as a biomarker in the screening for lung cancer using serum.

### EXPERIMENTAL EXAMPLE 4

Applications of CLSTN-1 in other cancer tissues were examined by the immunohistochemical staining method.

Multiple cancer tissue array, MTU39101 (purchased from Pantomic, USA), containing stage II melanoma was used to study the use of CLSTN-1 as a biomarker. Samples of normal lung tissue array without cancer were used as control groups.

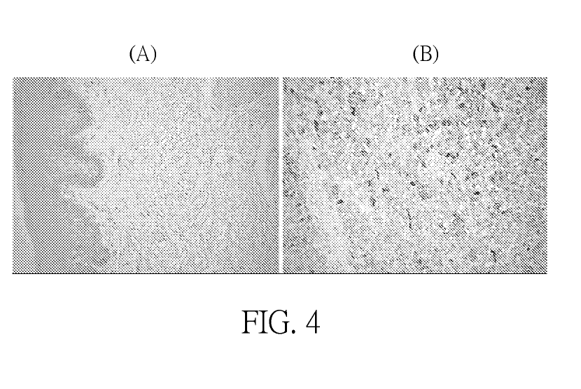

The lung tissue array was heated using a microwave, placed in an ethanol solution containing 2% H₂O₂, and fixed by 2% of BSA, 0.1% of Triton X-100 and 1% of sheep serum. The lung cancer tissue array and CLSTN-1 monoclonal antibodies were incubated at 37° C. for 2 hours, horseradish hydroperoxidase (HRP) (purchased from Biogenes, Germany) was added to bind to the CLSTN-1 monoclonal antibodies that formed complexes with the lung tissue arrays at room temperature for 30 minutes, and then 3,3-diaminobenzidine was added for imaging. Results are shown in FIG. 4.

As shown in FIG. 4, CLSTN-1 was expressed profusely in melanoma, but it was rarely expressed in normal skin tissues. Thus, CLSTN-1 can be used as a biomarker for detecting skin cancer.

### CONCLUSION

The aforesaid experimental examples elucidated that the expression levels of CLSTN-1 in the blood samples obtained from the patients with early cancer and the healthy volunteers are significantly different, and therefore, CLSTN-1 has higher sensitivity than that of the conventional CEA as a biomarker. Further, the differences in the staining results of the aforesaid lung tissue array are positively correlated with the severity degree of lung cancer. In addition to lung cancer, the aforesaid examples also illustrate that CLSTN-1 can be applied to detection of other cancers (such as skin cancer).

In light of the aforesaid illustrations, CLSTN-1 can be used as a biomarker for monitoring the therapeutic efficacy of a therapy or a medicament for treating a cancer patient. Based on the aforesaid features, CLSTN-1 can also be used for identifying efficacy of a medicament in the treatment of a cancer.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

1. A method for detecting cancer, comprising the steps of:
(1) obtaining a test sample of body fluid from a subject;
(2) measuring a calsyntenin-1 (CLSTN-1) level in the test sample of body fluid in vitro; and
(3) determining if the CLSTN-1 level in the test sample of body fluid is significantly higher than a normal CLSTN-1 level in a sample of body fluid from a healthy population of the same species as the subject, and if yes, indicating that the subject has the cancer.

2. The method of claim 1, wherein the cancer is lung cancer.

3. The method of claim 1, wherein the cancer is skin cancer.

4. The method of claim 1, wherein the subject is human.

5. The method of claim 1, wherein the body fluid is serum.

6. The method of claim 1, wherein the CLSTN-1 level in the test sample of body fluid in step (2) is measured by Western blot analysis or enzyme immunoassay.

7. A method for monitoring therapeutic efficacy of a therapy or a medicament for treating a cancer patient, comprising the steps of:
(1) obtaining a plurality of samples of body fluid from the cancer patient at one or more time points before and after applying the therapy or the medicament;
(2) measuring a plurality of CLSTN-1 levels in the plurality of samples of body fluid in vitro;
(3) comparing the plurality of CLSTN-1 levels in the plurality of samples of body fluid to one another; and
(4) determining if the plurality of CLSTN-1 levels in the plurality of samples of body fluid after applying the therapy or the medicament are significantly lower than the plurality of CLSTN-1 levels in the plurality of samples of body fluid before applying the therapy or the medicament, and if yes, indicating that the therapy or the medicament is effective for treating the cancer patient.

8. The method of claim 7, wherein the cancer patient is a human cancer patient.

9. The method of claim 8, wherein the body fluid is serum.

10. The method of claim 7, wherein the plurality of CLSTN-1 levels in the plurality of samples of body fluid in step (2) are measured by Western blot analysis or enzyme immunoassay.

11. A method for identifying efficacy of a medicament in treatment of cancer, comprising the steps of:
(1) selecting a group of mammals having cancer;
(2) obtaining a plurality of samples of body fluid from the group of mammals at one or more time points before and after applying the medicament;
(3) measuring a plurality of CLSTN-1 levels in the plurality of samples of body fluid; and
(4) determining if the plurality of CLSTN-1 levels in the plurality of samples of body fluid after applying the medicament are significantly lower than the plurality of CLSTN-1 levels in the plurality of samples of body fluid before applying the medicament, and if yes, indicating that the medicament is effective in the treatment of the cancer.

12. The method of claim 11, wherein the body fluid is serum.

13. The method of claim 11, wherein the plurality of CLSTN-1 levels of the plurality of samples of body fluid in step (3) are measured by Western blot analysis or enzyme immunoassay.

14. A kit suitable for use in the method of claim 1, comprising:
a molecule capable of recognizing CLSTN-1 or an unique segment of the CLSTN-1; and
a container or a solid support for carrying the molecule.

15. The kit of claim 14, further comprising a reagent for detecting the molecule capable of recognizing the CLSTN-1 or the unique segment of the CLSTN-1.

16. The kit of claim 14, further comprising an instruction booklet for using the kit.

17. The kit of claim 14, wherein the molecule capable of recognizing the CLSTN-1 or the unique segment of the CLSTN-1 is a CLSTN-1 monoclonal antibody.

18. The kit of claim 15, wherein the molecule capable of recognizing the CLSTN-1 or the unique segment of the CLSTN-1 is a CLSTN-1 monoclonal antibody, and the reagent for detecting the CLSTN-1 monoclonal antibody is a CLSTN-1 secondary antibody linked to horseradish peroxidase (HRP).

19. An application of CLSTN-1 as a biomarker in a method for detecting cancer.

20-21. (canceled)

22. A method for monitoring therapeutic efficacy of a therapy or a medicament for treating a a cancer patient, comprising:
using CLSTN-1 as a biomarker.
